(19) United States
(12) Patent Application Publication
(10) Pub. No.: US 2023/0010728 A1
(43) Pub. Date: Jan. 12, 2023

(54) Device and Method for Centrifuging a Physiological Fluid

(71) Applicant: Cervos Medical LLC, Marshfield, MA (US)

(72) Inventor: Andrew McGillicuddy, Humarock, MA (US)

(21) Appl. No.: 17/859,815

(22) Filed: Jul. 7, 2022

Related U.S. Application Data
(60) Provisional application No. 63/219,137, filed on Jul. 7, 2021.

(51) Int. Cl. A61M 1/36

(52) U.S. Cl. CPC A61M 1/3693; A61M 2202/0427

(57) ABSTRACT

A device and associated method for centrifuging a physiological fluid includes a container including a tip, a base, and a barrel extending between the tip and the base to hold a physiological fluid. The container includes a plunger positioned within the barrel of the container. The plunger includes a concave collection surface that faces the tip of the container. A plunger gasket seal is in sealing engagement with an inside wall of the barrel of the container. A bottom support is provided to support the base of the container at least partially within the bottom support for use in a centrifuge. The container is coupled to the bottom support using an interference fit. The collection surface of the plunger and at least the barrel can be made of the same material. Advantageously, the device can be used to process blood without the need for any added anticoagulant in the blood.

### RELATED APPLICATION

This application claims the benefit of U.S. Provisional Application No. 63/219,137, filed on Jul. 7, 2021. The entire teachings of the above application are incorporated herein by reference.

### BACKGROUND

Increasingly plastic surgeons are using aspirated fat, platelet rich plasma (PRP), marrow, and other biologics that are harvested at the point of care and are centrifuged to create a filler material for face-lifts and other cosmetic surgeries, such as hand and breast augmentation. The processed fat is often referred to as a fat graft procedure. The aspirated tissue is often aspirated into a syringe which is then centrifuged. Surgeons are interested in fractionating the tissue at point of care and using the various fractions to assist in the surgery or procedure. Typically, different devices are required to fractionate marrow, blood, and fat because the characteristics of the tissues are so different. For example, fat is much less dense than blood. Fat has a large amount of oil while marrow has typically little to none. Blood has a larger range of hematocrit. Fat typically has no hematocrit. Marrow has more nucleated cells than blood. Often, clinicians need to open separate kits to process fat, blood, and marrow.

Therefore, a need exists for a single system that can be used to create the different fractions of tissue that clinicians desire.

### SUMMARY

Devices and associated methods for centrifuging a physiological fluid are provided. In general, a device includes a syringe-like container including a plunger positioned with the container, the contained being supported at its base by a bottom support structure and supported at its tip by a top support structure for use in a centrifuge.

Centrifuging fluid in a container can cause the container to leak, deform or both. For example, typically a tube is centrifuged at between 50 g and 1000 g force. A need exists to centrifuge material simply without the material leaking and to provide a means to further transfer and use the separated material. Embodiments of the present invention provide convenient and novel tools to centrifuge various tissues that are routinely aspirated during point of care procedures. These tissues typically fall under the category of adipose tissue, blood, marrow, or combinations thereof. These tissues are encompassed by the terms “biological fluid” and “physiological fluid,” which are used interchangeably herein.

A device for centrifuging a physiological fluid includes a container including a tip, a base, and a barrel extending between the tip and the base to hold a physiological fluid. The container includes a plunger positioned within the barrel of the container. The plunger includes a concave collection surface that faces the tip of the container. The plunger further includes a plunger gasket in sealing engagement with an inside wall of the barrel of the container. A bottom support is provided to support the base of the container at least partially within the bottom support for use in a centrifuge. A top support can be provided to support the tip of the container at least partially within the top support for use in the centrifuge. The container is coupled to the top support (if present) and bottom support structures using an interference fit.

The device can further include a screen positioned in the barrel of the container, e.g., between the plunger and the tip of the container. The screen can have a circumference such that it creates a press fit between the inside wall of the barrel and the screen when inserted into the barrel.

The bottom support can include a support base configured to provide access to the plunger. For example, the support based can have a hole, which may be centrally located in the base. The hole can be threaded and configured to accept a screw mechanism. The screw mechanism can be used to apply force to the plunger, e.g., to move the plunger within the barrel, such as to push the plunger toward the tip of the container.

The collection surface of the plunger and at least the barrel of the container can be made of the same material, preferably polypropylene. In some embodiments, the container is entirely made from polypropylene.

The container tip can include a luer port and the device can include a cap to close the luer port. A swabable luer valve can be coupled to the luer port. The cap can be configured to fit onto the swabable luer valve to close the luer port.

The barrel can include at least one protrusion and the bottom support can include at least one recess configured to receive the protrusion. The recess and the protrusion can cooperate to provide a snap fit engagement between the container and bottom support when coupled. The at least one protrusion of the barrel can be cantilevered, and the recess can be windowed to allow an external force to be applied to the protrusion to displace the protrusion from the recess, to cause disengagement of the snap fit.

The plunger, which can be substantially cylindrical in shape, can include a rounded top rim encircling the concave surface, a rounded bottom rim, and a sidewall extending between the top rim and bottom rim, the sidewall including an annular recess to hold the gasket seal.

The container can include an inner surface near the tip of the container, the inner surface arranged to face the concave surface of the plunger. The inner surface can be substantially flat or can be slightly concave but less concave than the concave collection surface of the plunger. In an embodiment, the inner surface is convex.

A method for centrifuging a physiological fluid includes holding a physiological fluid in a container, the container including a tip, a base, and a barrel extending between the tip and the base, the container including a plunger positioned within the barrel, the plunger including concave surface facing the tip of the container and a plunger gasket in sealing engagement with an inside wall of the barrel. The method further includes supporting the container at least partially within a bottom support, the container being coupled to the bottom support using an interference fit; and centrifuging the physiological fluid in the container supported by bottom support.

The physiological fluid can be centrifuged with the base of the container away from a center of a centrifuge rotor (e.g., luer tip up) or with the tip of the container away from a center of a centrifuge rotor (e.g., luer tip down).

The method can include supporting the tip of the container with a top support at least partially within the top support, the container being coupled to the top support using an interference fit. The physiological fluid can be centrifuged in the container supported by the top and bottom and supports.

The physiological fluid can be centrifuged in a first centrifugation with the base of the container away from a center of a centrifuge rotor. The method can include extracting a first fraction of the physiological fluid from the container after the first centrifugation. A portion of the physiological fluid can be further centrifuged in a second centrifugation with the tip of the container away from a center of a centrifuge rotor.

The method can include extracting second and third fractions of the physiological fluid from the container after the second centrifugation.

In one example, the physiological fluid is adipose aspirate, the first fraction is a lipid layer, the second fraction is infranatant, and the third fraction is adipose graft tissue.

The container tip can include a luer port, in which case the method can further include closing the luer port with a cap prior to the second centrifugation.

The method can include, after the first centrifugation, transferring the first fraction of the physiological fluid to a second container. The second container can include a tip, a base and a barrel extending between the tip and the base, the second container further including a plunger positioned within the barrel, the plunger including a concave collection surface facing the tip of the second container and a gasket seal in sealing engagement with an inside wall of the barrel. The method can further include, in a second centrifugation, centrifuging the first fraction of the physiological fluid in the second container supported by a second bottom support. The method can further include extracting second and third fractions of the physiological fluid from the second container after the second centrifugation.

In one example, the physiological fluid is blood, the first fraction is plasma, the second fraction is platelet poor plasma (PPP), and the third fraction is platelet rich plasma (PRP).

When the physiological fluid is blood, the blood can be centrifuged without adding anticoagulant to the blood prior to centrifugation.

The method can include positioning a screen in the barrel of the container between the plunger and the tip of the container.

In an example where the bottom support includes a support base having a hole to provide access to the plunger of the container, the method can further include applying a mechanical force to the plunger with a screw mechanism threaded through the hole, the applied mechanical force assisting in extracting a fraction of the physiological fluid from the container.

A method for centrifuging a physiological fluid using the device includes loading the physiological fluid into the container of the device, and centrifuging the physiological fluid in the container supported by the bottom support of the device.

In one example, the physiological fluid is blood, in which case the blood can be centrifuged without adding anticoagulant to the blood prior to centrifugation.

Advantageously, a portion or all of the container and the plunger surface that is concave and comes in contact with biological fluid can be made of a material that does not activate blood clotting factors. A suitable material is polypropylene.

By making the surface of the plunger the same material as the container barrel, e.g., polypropylene, the blood only comes in contact substantially with polypropylene. This allows processing of the blood without a need for any anti-coagulant in the blood to keep the blood from clotting. Effectively, the blood does not clot during processing in the centrifuge even though no external anti-coagulant has been used. Polypropylene is an inert plastic material that does not activate clotting factors in blood during the processing time, e.g., from the time the blood is drawn and centrifuged until time the processed blood is used to treat the patient. Processing time can be from about one hour to about two hours. Materials other than polypropylene may be used, provided that the materials have the desired properties described above.

Some commercial systems are made of material other than polypropylene or have an insert to help separation in the container that is made of a different material or have an elastomer plunger made of different material. Often, these different materials activate blood during centrifugation. Also, some blood separation kits have a dual purpose, where the second purpose is to send samples to a laboratory, so the blood is not tested for hours and needs to be treated with anticoagulant. This contrasts with applications, where the blood is obtained from a patient to test or treat the patient almost immediately after processing of the blood.

In one embodiment, a method includes centrifuging blood in a first container, decanting plasma, transferring the plasma to a second container, and centrifuging a second time to create a concentrate, e.g., platelet rich plasma, all without the need for anticoagulant.

Since it is common practice to always work with anticoagulated blood, several prior approaches may have omitted using anticoagulant in their descriptions because it is assumed that one needs to add anticoagulant to the blood for processing. Commercial systems on the market that use a two-spin method or apheresis method are believed to use anticoagulant. Advantageously, embodiments of the present approach do not need anticoagulant when performing a two-step centrifuge process to make platelet rich plasma from blood.

Embodiments of the device and method can be used to process blood without adding anticoagulant. When processing blood without anticoagulant, centrifugation is performed at less than 2000 g force and for a duration of less than 15 minutes.

A common anticoagulant for blood collection for use with apheresis devices is Anticoagulant Citrate Dextrose Solution USP (ACD) Solution A, also referred to as ACD-A.

Embodiments can be used for safe and rapid preparation of autologous platelet rich plasma from a sample of a patient's peripheral blood at the patient's point of care. Preparation of the platelet rich plasma can be performed for various uses, including mixing with autograft bone, allograft bone, or combinations thereof, to improve handling characteristics of bone graft.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A is a perspective view of a device for centrifuging a physiological fluid according to an example embodiment.

FIG. 1B is a side view of the device of FIG. 1A.

FIG. 1C is section view of the device of FIG. 1B.

FIG. 1D illustrates a filter (e.g., a screen) of the device of FIGS. 1A-1C.

FIG. 1E illustrates a sliding gasket plunger of the device of FIGS. 1A-1C.

FIGS. 1F, 1G, and 1H are side, section, and bottom views, respectively, of the plunger of FIG. 1E.

FIG. 1I is another section view of the plunger of FIG. 1E.

FIGS. 1J and 1K are side and section views, respectively, of the container of the device of FIGS. 1A-1C.

FIG. 2 is a perspective view of a screw mechanism received in a threaded access port of a bottom support according to an example embodiment.

FIGS. 3A-3E illustrate an example process of using the device of FIGS. 1A-1C for centrifuging a biological fluid, in particular, centrifuging fat aspirate (adipose aspirate), to separate the fat aspirate into component fractions.

FIG. 3A is a perspective view of the device prior to fat aspirate being loaded into the container of the device.

FIG. 3B is a perspective view of the device of FIG. 3A illustrating fat aspirate in the container separated into fractions following a first centrifugation step.

FIG. 3C is a perspective view of the device of FIG. 3B illustrating the device after a second centrifugation step, where the luer tip is closed with a cap and the device is placed in a centrifuge with the capped luer tip down. The fat graft is separated from the infranatant.

FIG. 3D is a perspective view of the device of FIG. 3C after the second centrifugation step. After removal of the cap, the infranatant can be extracted through the luer port from the upper portion of the container, leaving the fat graft behind.

FIG. 3E illustrates the device of FIG. 3D with a collection syringe coupled to the luer port of the container and a screw mechanism coupled to the bottom support. The fat graft is transferred from the container to the collection syringe with mechanical assistance from the screw mechanism and filtered through a screen positioned within the container.

FIG. 4A is a perspective view of a kit including a device for centrifuging a biological fluid and a syringe for transferring fluid into or out of the container of the device. The device includes a plunger positioned in the container and a bottom support coupled to the base of the container.

FIG. 4B is perspective view of the plunger of the device of FIG. 4A.

FIGS. 5A-5E illustrate an example process of using more than one device of FIG. 4A for centrifuging a biological fluid, in particular blood, to separate the blood into component fractions.

FIG. 5A is a perspective view, prior to first centrifugation, of a first device including a first container and a first bottom support. Also illustrated is a first syringe suitable for loading blood into the container.

FIG. 5B is a perspective view, post first centrifugation, of the device of FIG. 5A, with cloudy plasma being separated from infranatant. Also illustrated is a second syringe, e.g., a collection syringe, that can be used to extract the plasma from the container of the device.

FIG. 5C is a perspective view, post first centrifugation but prior to second centrifugation, of cloudy plasma loaded into a second device including a second container supported by a second bottom support. Also illustrated is the second syringe, which can be used to load the plasma into the container of the second device.

FIG. 5D is a perspective view, post second centrifugation, of the second device illustrating a layer of platelet poor plasma (PPP) above a layer of platelet rich plasma (PRP) above the plunger. Also illustrated is a third syringe, e.g., a collection syringe, that can be used to extract the platelet poor plasma from the container of the device.

FIG. 5E is a perspective view of the second device, post second centrifugation and after extraction of the platelet poor plasma illustrating the cell collection surface of the plunger. Platelet rich plasma (PRP) can be extracted through the luer port using a fourth syringe, which is also illustrated.

FIG. 6 illustrates a kit for processing blood that includes a device for centrifuging a biological fluid according to an example embodiment.

FIG. 7 illustrates an example process of using the device of FIG. 6 to obtain platelet rich plasma (PRP) from blood using a single centrifugation step (single spin).

FIG. 8 illustrates another kit for processing blood that includes two devices for centrifuging a biological fluid according to an example embodiment.

FIG. 9 illustrates an example process of using the devices of FIG. 8 to obtain platelet rich plasma (PRP) from blood using two centrifugation steps (double spin).

FIG. 10 illustrates another kit for processing blood that includes three devices for centrifuging a biological fluid, which allow for larger volumes of the fluid to be processed, according to an example embodiment.

FIG. 11 illustrates an example process of using the devices of FIG. 10 to obtain platelet rich plasma (PRP) from blood using two centrifugation steps (double spin).

### DETAILED DESCRIPTION

A description of example embodiments follows.

The device described herein is an improvement over the device described in International Application No. PCT/US2018/063317, entitled “Apparatus And Method For Centrifuging a Biologic,” filed on Nov. 30, 2019 and published Jun. 6, 2019 as WO 2019/108937, the teachings of which are incorporated herein by reference in their entirety.

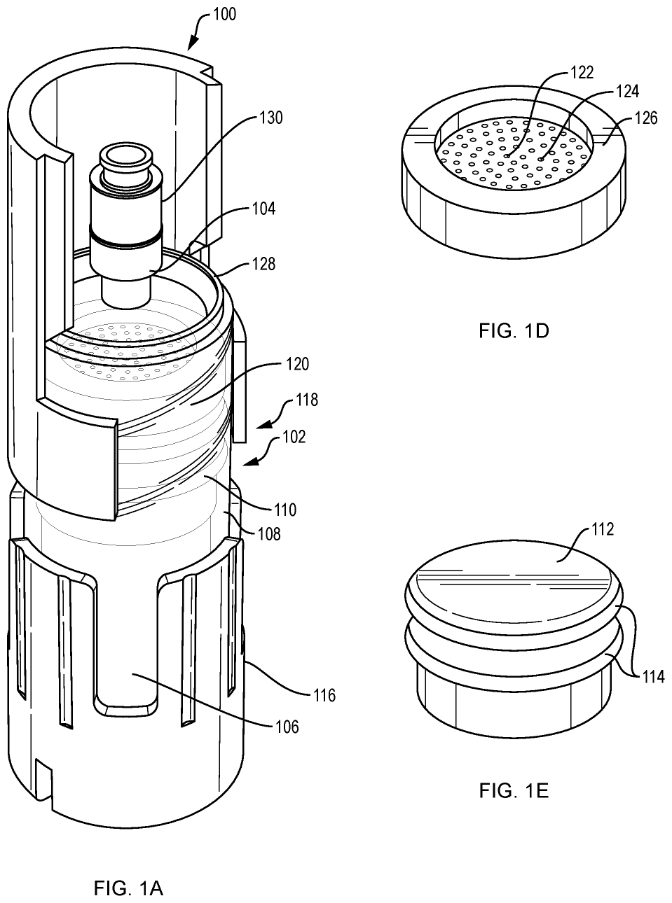

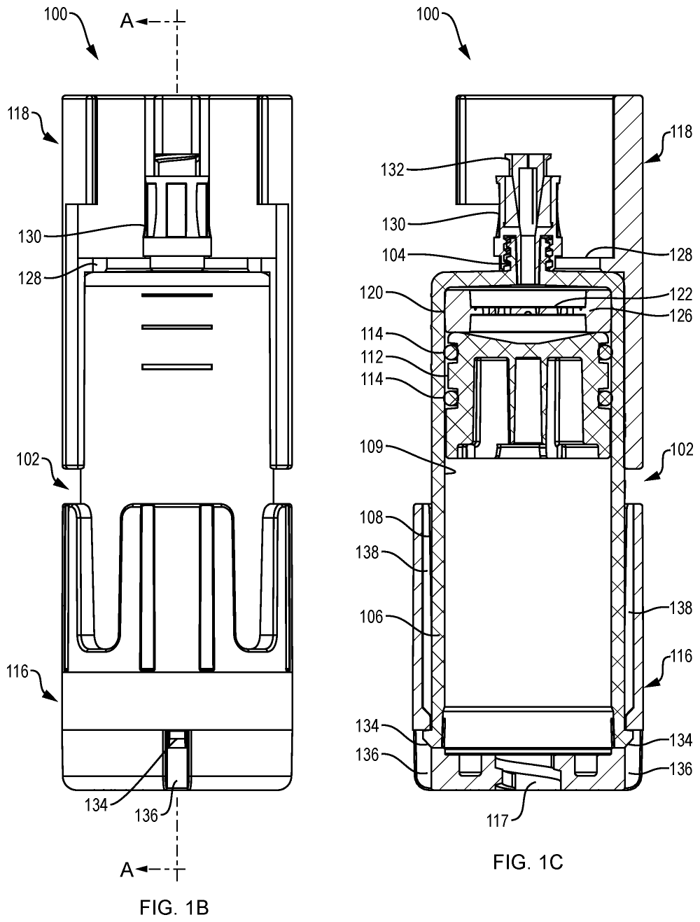

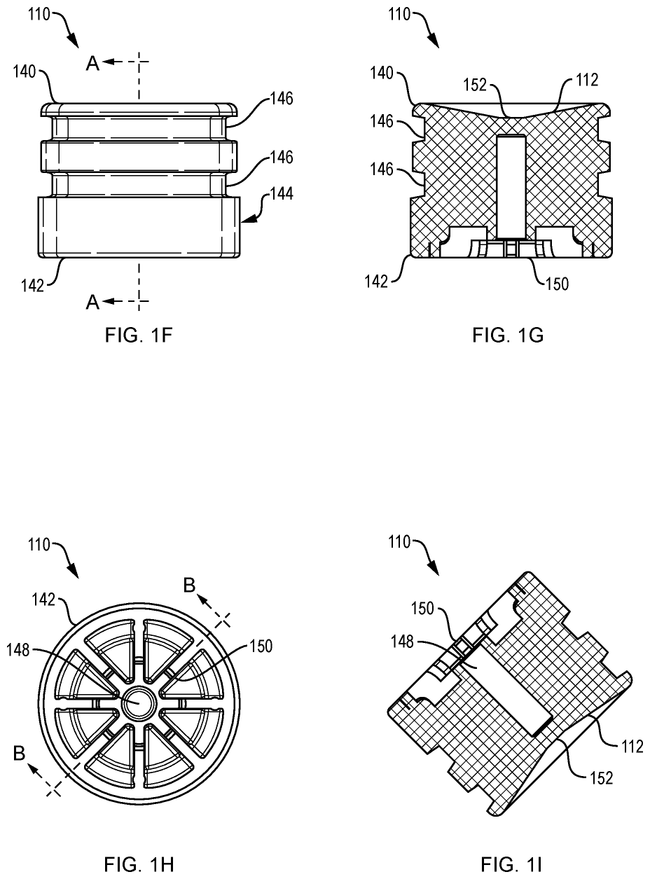

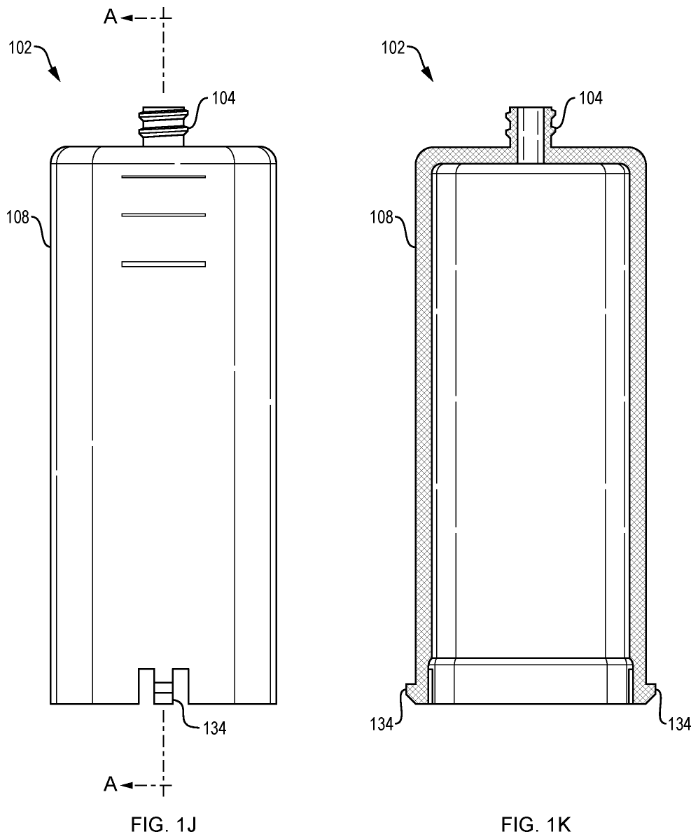

FIGS. 1A-1C illustrate a device 100 for centrifuging a physiological fluid. The device 100 includes a container 102 including a tip 104, a base 106, and a barrel 108 extending between the tip 104 and the base 106, to hold a physiological fluid. A plunger 110 is positioned within the barrel 108 of the container 102 and includes a concave collection surface 112 that faces the tip 104 of the container. The plunger further includes at least one plunger gasket 114 in sealing engagement with an inside wall 109 (FIG. 1C) of the barrel 108 of the container 102. A bottom support 116 is provided to support the base 106 of the container at least partially within the bottom support for use in a centrifuge. A top support 118 is optionally provided to support the tip 104 of the container at least partially within the top support for use in the centrifuge. The container 102 is coupled to the top support (if present) and bottom support structures using an interference fit.

The tip 104 of the container 102 can include or otherwise incorporate a luer connector. As illustrated, a luer lock valve 130 can be coupled to the tip 104 of the container to allow for controlled fluid transfer into and out of the container. The valve 130 can provide a swabable valve port 132 (see, e.g., FIG. 1C).

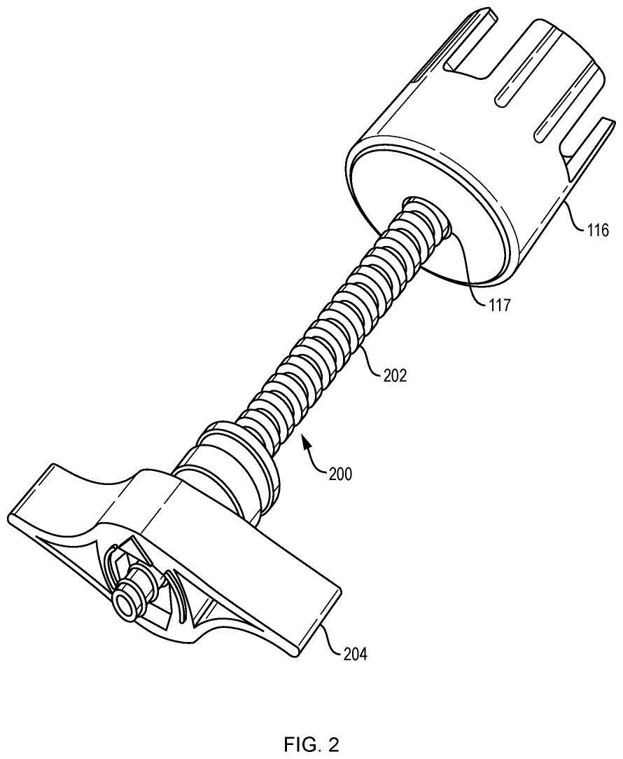

The device and its components, as illustrated in FIGS. 1A-1E and FIG. 2, include several features that provide improvements of prior approaches, such features include:

1) The plunger 110, which is movably positioned in the barrel 108 of the container, has a concave surface 112 that facilitates the collection of target cells after centrifugation.

2) The thickness of the walls of the barrel 108 can be 2 mm or greater. The wall thickness provides strength and rigidity to the container, reducing the risk that the container wall will deform under high g-force loads during centrifugation.

3) The barrel 108 and the plunger 110 can be made of polypropylene.

4) The seal 114 of the plunger can be in the form of a gasket, which can be an O-ring or an X-ring. An X-ring gasket is a standard part and known in industry. There can be more than one gasket, e.g. two gaskets as illustrated in FIG. 1E.

5) A screen 120 (FIG. 1D) can be press fit into the barrel 108 so that at g-forces up to 2,000, the screen will not slide or move in the barrel.

6) A bottom support structure 116 can be press fit over the bottom of the container barrel 108. As illustrated, at least a portion of the bottom support structure 116 encompasses the entire 360 degrees of the circular circumference of the barrel 108.

7) As illustrated in FIGS. 1C and 2, the bottom support structure 116 can have a threaded center hole 117, at the base of the support structure, that is configured to accept a screw mechanism 200. This arrangement can provide a mechanical assist to facilitate fluid transfer between the container and another device, e.g., a syringe. In the example shown in FIG. 2, the screw mechanism 200 includes an elongated screw 202 that extends from a handle 204.

8) A top support structure 118 (FIGS. 1A-1C) can be press fit over the top of the container barrel 108. The top support structure 118 preferably does not encompass the entire 360 degrees of the circular circumference of the barrel 108. The top support structure has a feature 128 (e.g., a lip) that protrudes inward and that has an interference with the top of the barrel 108 such that when the assembled device is centrifuged with the luer tip down, the force due to centrifugation is against the top support structure and outer barrel and not the center luer tip.

9) The bottom and top support structures allow the assembled device 100 to be centrifuged in either orientation, luer tip up or luer tip down.

A user can load fluid into the vessel, e.g., the vial or container 102, through the luer tip 104 and past the screen 120 that is press fit in place. The user can centrifuge the fluid material in the vessel in either direction, luer up or luer down. The support structures (116, 118) absorb the forces of centrifugation to prevent the assembly from breaking or leaking. The fluid can be evacuated from the barrel of the container through the screen using the luer connection. Additional force can be applied to the plunger 110 by using the screw feature 200 (FIG. 2) to allow more viscous materials to be forced through the screen and out of the barrel. Certain versions of the device can be provided with a screen and others without the screen. The side of the plunger 110 that is facing the luer tip 104 includes a collection surface 112 that is concave to better concentrate the target cells/densest material in a smaller location.

As illustrated in FIGS. 1B and 1C, the barrel 108 can include at least one protrusion 134 and the bottom support 116 can include at least one recess 136 configured to receive the protrusion 134. In the example shown, there are two protrusions 134 that correspond to two recesses 136 in bottom support 116. The recess 136 and the protrusion 134 can cooperate to provide a snap fit engagement between the container 102 and bottom support 116 when the two are coupled. Each protrusion 134 of the barrel can be cantilevered, as further illustrated in FIGS. 1J-1K, and the recess 136 can be windowed to allow an external force to be applied to the protrusion to displace the protrusion from the recess, to cause disengagement of the snap fit. The bottom support 116 includes longitudinal channels 138, which are aligned with recesses 136, to guide the protrusions 134 toward the corresponding recesses 136 during assembly of the container 102 and the bottom support 116.

As illustrated in FIG. 1D, the screen 120 includes a perforated plate 122 having a plurality of holes 124 for fluid passage. The plate 122 is surrounded by a ring 126 whose thickness is greater than the thickness of the plate.

The screen 120 can serve two functions. It can force laminar flow during extraction of the separated layers (e.g., separated fractions) after centrifugation. Laminar flow can reduce or prevent mixing of the separated layers. This enables removal of different density layers to capture high density fat. In addition, the screen serves as sieve, prevent larger particles, e.g., larger adipose particles from exiting the container. This can reduce or prevent clogging of the container tip, e.g., during extraction from the container. Embodiments provide a closed system that allows for removal of excess lipids and blood with a screen and a mechanical assist fluid transfer feature that micronizes the adipose graft for easy application. The system is considered closed because there is not vent and no port other than at the luer tip. The port at the luer tip can be capped, as further described herein.

FIGS. 1F-1I are additional views of gasket plunger 110 of the device 100 for centrifuging a biological fluid. The plunger 110, which can be substantially cylindrical, includes a rounded top rim 140 encircling the concave surface 112 and a rounded bottom rim 142. The radius of curvature of the top rim 140 can be twice that of the radius of curvature of the bottom rim 142. In one example, the radius of curvature of the top rim is 0.050 inches and that of the bottom rim is 0.025 inches. A sidewall 144 extends between the top rim and bottom rim. The sidewall includes or defines at least one annular recess or groove 146 that is configured to hold a gasket seal 114. Here, the sidewall includes two recesses 146, which are spaced apart, the top recess close to the rounded top rim. The recesses 146 can be equal in height and depth and the spacing between the recesses 146 can be greater than the height of each of the recesses.

The plunger's concave surface 112 forms a central depression 152. The depth of the central depression can be less than the width and/or the diameter of the plunger. For example, the depth of the central depression 152 can be 0.080 inches and the diameter of the plunger can be 0.141 inches.

As illustrated in FIGS. 1G-1I, the plunger 110 includes central bore 148, which extends from an opening at the bottom of the plunger to below the top surface of the plunger, there being no opening at the top of the plunger. Plural spokes 150 extend radially from the region of the central bore 148 of the plunger toward the sidewall 144 of the plunger. The spokes 150 provide structural support to the sidewall and upper surface of the plunger, and guard against deformation or structural failure that otherwise could result from exposure to high g-forces during centrifugation.

FIGS. 1J and 1K illustrate the container 102 of the device 100. The container 102 includes barrel 108 extending from the tip 104 of the container. The barrel 108 includes at least one protrusion 134 configured to engage with at least one recess 136 of the bottom support 116. As illustrated, the at least one protrusion 134 is cantilevered. The protrusion 134 and the recess 136 can cooperate to provide a snap fit engagement between the container 102 and bottom support 116 when the two are coupled, thereby locking the container 102 to the bottom support 116. The container 102 includes an inner surface near the tip 104 of the container, the inner surface arranged to face the concave surface of the plunger. The inner surface can be substantially flat or can be slightly concave, as illustrated, but less concave than the concave collection surface of the plunger. In an embodiment (not shown), the inner surface is convex.

Further details regarding the use of the device, including additional figures, are provide in the Examples below, which illustrate additional views and alternative embodiments.

### EXEMPLIFICATION

### Example 1—Processing Adipose Aspirate

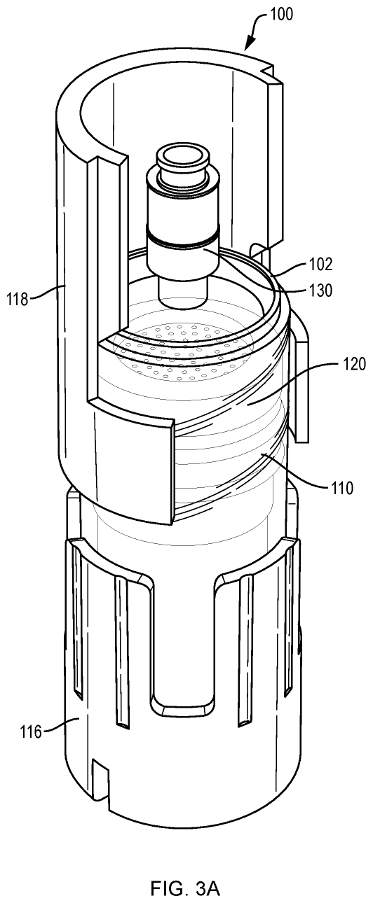

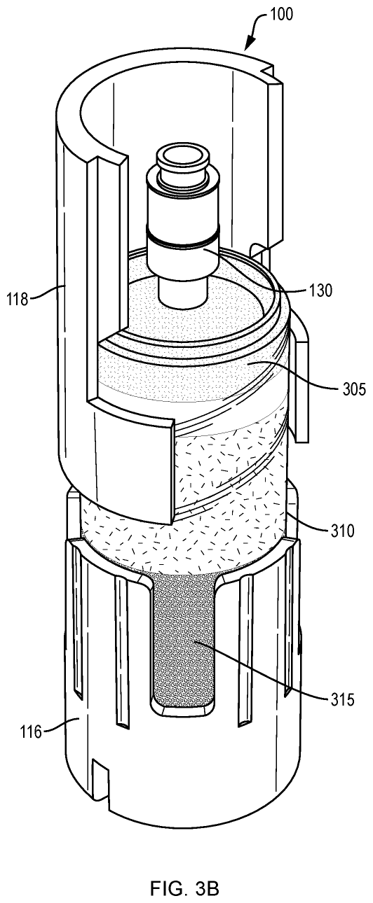

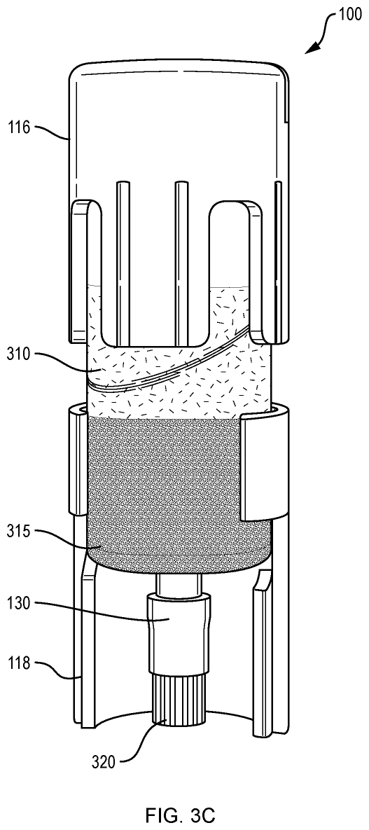

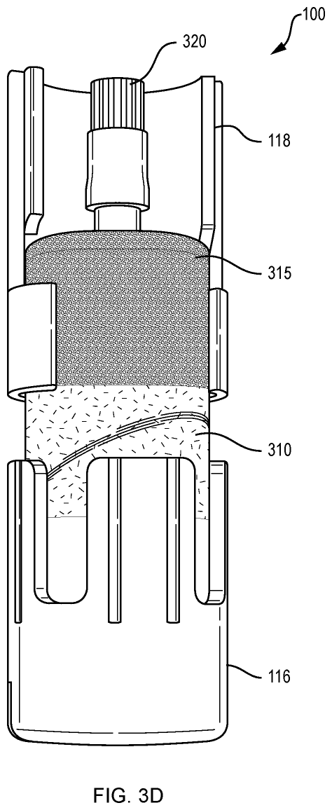

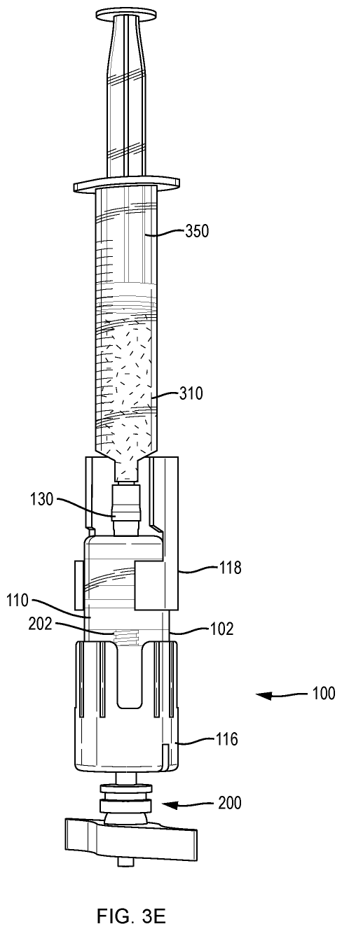

FIGS. 3A-3E illustrate an example process of using the device 100 of FIGS. 1A-1C for centrifuging a biological fluid, in particular, for centrifuging fat aspirate (adipose aspirate) using a standard centrifuge, to separate the fat aspirate into component fractions.

Step 1 (FIG. 3A)

Fill container 102 of device 100 with 30-40 cc of adipose aspirate.

Place device 100 into centrifuge (swingout rotor bucket). Use a counter-balance or an additional disposable device to balance the centrifuge.

Swab port valve 130 (to remove any residual blood/aspirate).

Centrifuge at 3200 RPM for 2 minutes.

Step 2 (FIG. 3B)

Remove device 100 from the centrifuge bucket and swab port valve 130.

Decant lipid layer 305 to a syringe. Stop decanting lipid layer 305 when adipose graft 310 interface enters the neck of luer tip below port valve 130, leaving the adipose graft 310 and infranatant 315 in container.

Step 3 (FIG. 3C)

Attach a locking cap 320 to port valve 130.

Place device 100 in rotor bucket (luer tip down). Use a counter-balance or an additional disposable device to balance the centrifuge.

Centrifuge at 3200 RPM for 1 minute.

Step 4 (FIG. 3D)

Remove device 100 from centrifuge bucket.

Remove cap 320.

Decant infranatant 315 with a syringe. Stop decanting infranatant when the adipose graft 310 appears in neck of valve 130.

Step 5 (FIG. 3E)

Connect collection syringe 350 to leer valve 130 and connect mechanical assist device 200 to bottom support 116.

Transfer the adipose graft 310 from the container 102 to the syringe 350 using threaded assist device 200. The screw 202 of device 200 contacts and pushes the plunger 110, to extract the adipose graft. If a screen 120 is present in the container, the adipose grab is filtered through the screen.

### Example 2—Processing Blood

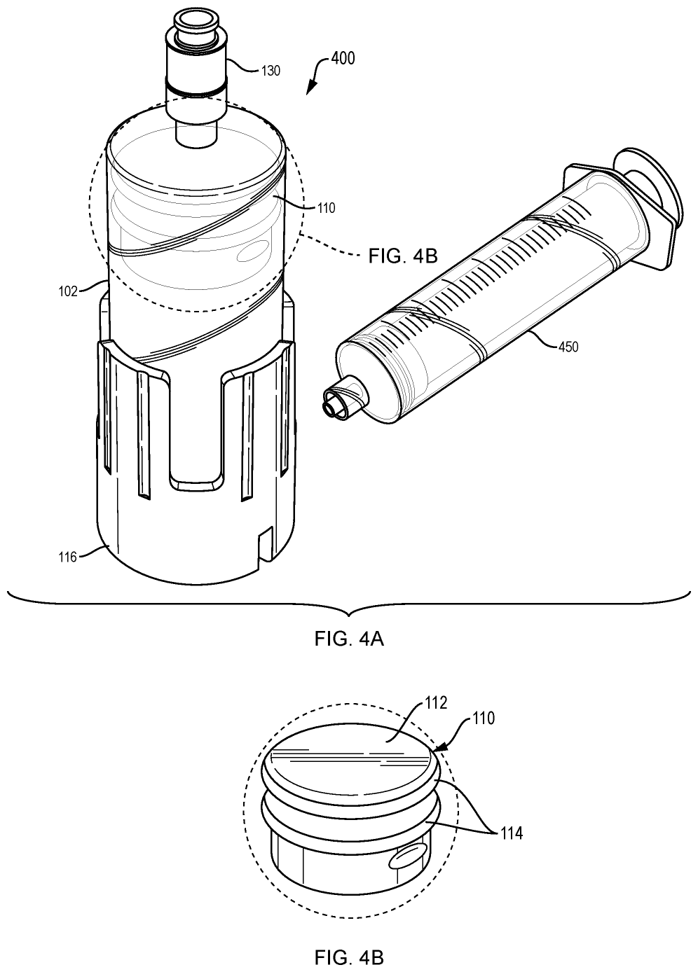

FIG. 4A is a perspective view of a kit including a device 400 for centrifuging a biological fluid and a syringe 450 for transferring fluid into or out of container 102 of the device 400. The device 400 is substantially similar to device 100, except that device 400 does not include a screen or an upper support, which are typically not required for processing blood. The device 400 includes a plunger 110 positioned within the container 102 and a bottom support 116 coupled to the base of the container 102. The container and bottom support are configured to create a tight fit, at least at a base portion of the container, to hold the container filled with the biological fluid so that the plunger 110 is supported within the container to prevent any leaking of fluid around the plunger, e.g., between the plunger and an inside wall of the container.

The plunger's platelet collection surface allows for maximum volume reduction, lowest hematocrit, highest platelet recovery, and highest increase over baseline, all in an easy to use and disposable closed system.

As illustrated in FIG. 4B, a key feature of the plunger 110 is platelet collection surface 112. The concave surface 112 efficiently collects platelets allowing for maximum volume reduction and customizable increases over baseline. The plunger 110 includes two sealing gaskets 114 to seal against the inner wall of the container 102 while allowing up and down movement of the plunger within the container 102.

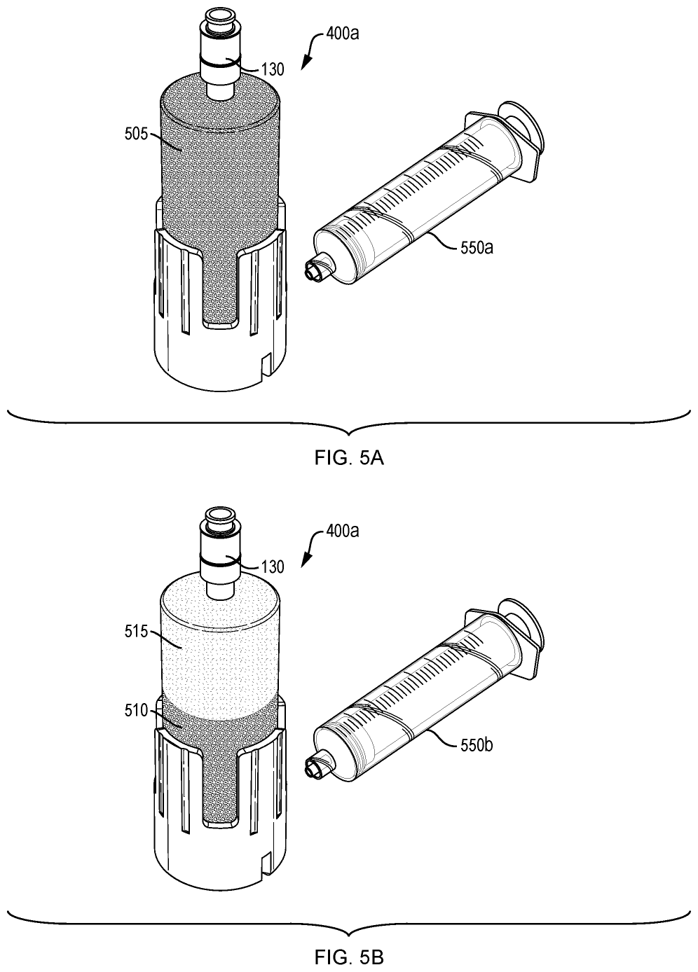

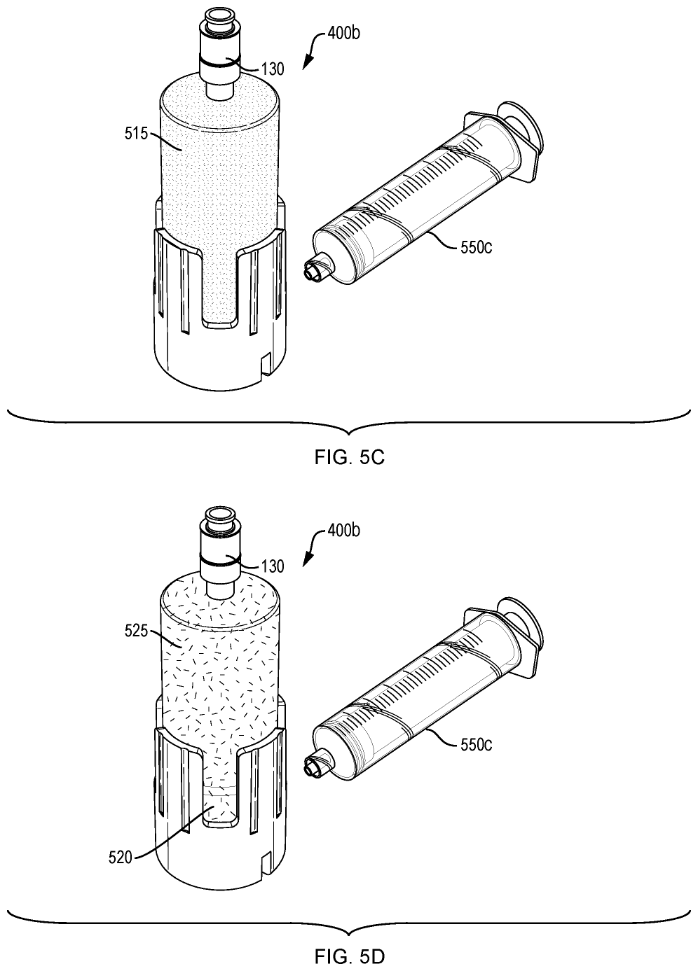

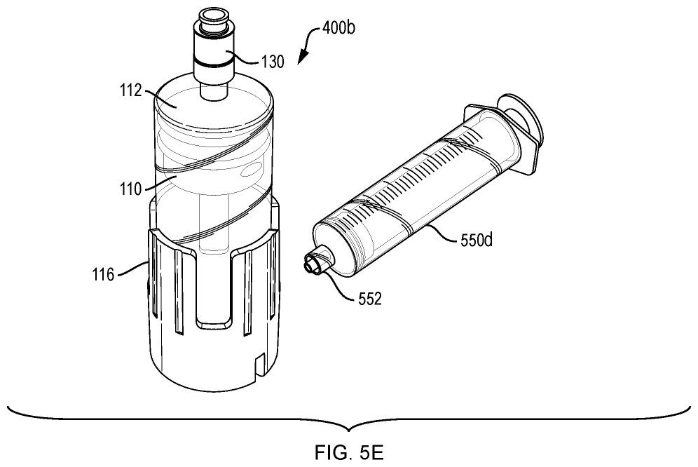

FIGS. 5A-5E illustrate an example process of using two devices 400a, 400b, such as device 400 of FIG. 4A, for centrifuging and separating blood into component fractions. The devices are configured for use with a standard centrifuge.

FIG. 5A is a perspective view, prior to first centrifugation, of a first device 400a including a first container and a first bottom support. Also illustrated is a first syringe 550a suitable for loading blood into the container of the device.

Step 1 (FIG. 5A)

Fill container of device 400a with 30 cc of blood. The blood may be anti-coagulated but need not be.

Place device 400a into a centrifuge (swingout rotor bucket) and use counterweight or additional disposable device with equal weight.

Swab port valve 130 (to remove any residual blood).

Centrifuge at 3200 RPM for 2 minutes.

Step 2 (FIG. 5B)

Remove device 400a from centrifuge bucket and swab port valve 130.

Decant cloudy plasma 515 to syringe 550b.

Stop decanting plasma when red blood cell (RBC) interface, e.g., infranatant 510, enters neck of luer tip of the container.

Step 3 (FIG. 5C)

Load second device 400b with cloudy plasma 515 from syringe 550b. Optionally, add 2 ml anti-coagulant to the second device 400b prior to loading the plasma.

Place second device 400b into centrifuge (swingout rotor bucket) and use counterweight or additional disposable device with equal weight.

Swab port valve 130.

Centrifuge at 3200 RPM for 6 minutes.

Step 4 (FIG. 5D)

Remove device 400b from the centrifuge bucket. Platelets, of platelet rich plasma 520, are expected to be visible on collection surface of the plunger.

Decant upper, clear plasma 525 with syringe 550c. Stop decanting plasma at desired level as PRP collection surface of plunger approaches the top of the device 400b.

Step 5 (FIG. 5E)

Decant remaining plasma and platelets, e.g., platelet rich plasma 520, with syringe 520d, flushing back and forth to resuspend platelets as needed. As exemplified by syringe 550d, the syringe includes a male luer port 552, which can couple to port valve 130, thereby allowing for needleless transfer of fluid. The needless design is a feature of all devices and syringes described herein, including devices 100, 400, and 400a-b, and syringes 350, 450, and 550a-d.

### Example 3—Processing Blood, Single Spin

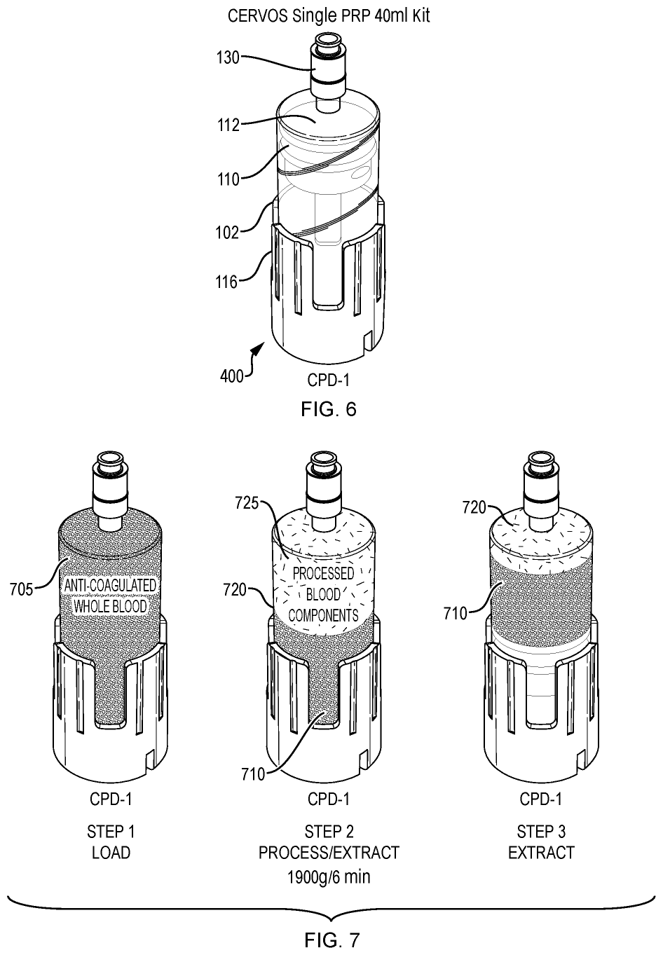

FIG. 6 illustrates a kit for processing blood that includes a device 400 (CPD-1) for centrifuging a biological fluid. As disclosed herein, the device 400 includes a container 102 supported by bottom support 116, a plunger 110 having a concave collection surface 112 and being disposed within the container 102, and a luer lock valve 130.

FIG. 7 illustrates an example process of using the kit and device 400 of FIG. 6 to obtain platelet rich plasma (PRP) from blood using a single centrifugation step.

STEP 1: Load blood 705, optionally anticoagulated, into the device 400 (CPD-1).

STEP 2: Process blood into components infranatant 710, platelet rich plasma (PRP) 720, and platelet poor plasma (PPP) 725 by centrifuging at 1900 g for 6 minutes. Extract PPP 725 using a syringe.

STEP 3: Extract PRP 720 using a syringe.

### Example 4—Processing Blood, Double Spin

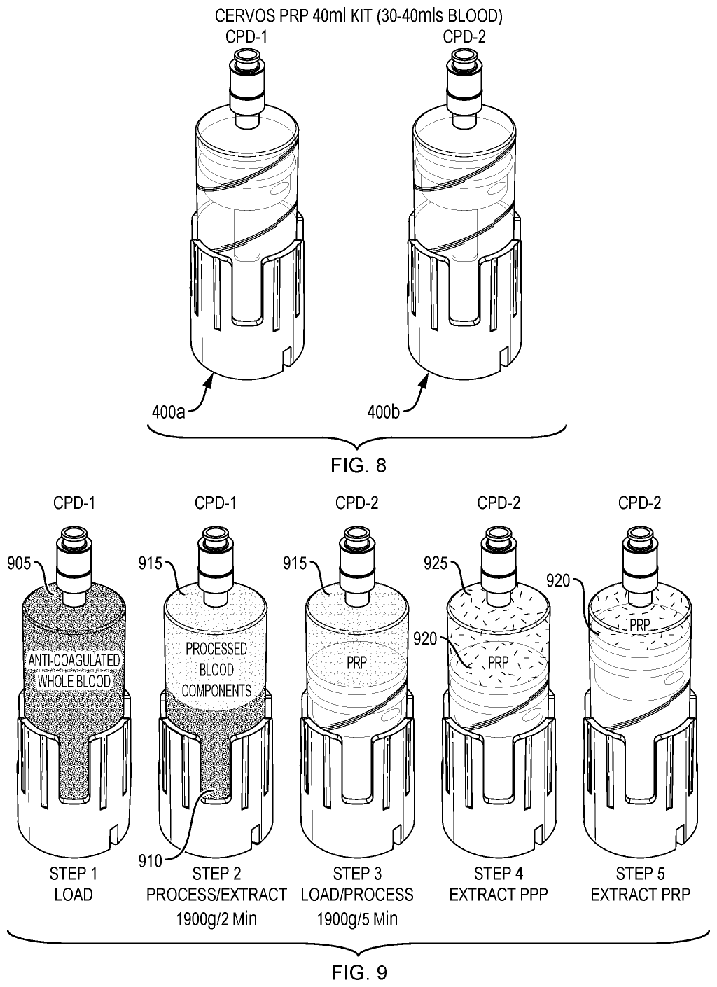

FIG. 8 illustrates another kit for processing blood that includes two devices, 400a (CPD-1) and 400b (CPD-2), for centrifuging a biological fluid. The kit is configured to centrifuge about 30 ml to about 40 ml of fluid such as blood.

FIG. 9 illustrates an example process of using the kit and devices of FIG. 8 to obtain platelet rich plasma (PRP) from blood using two centrifugation steps (double spin).

STEP 1: Load blood 905, optionally anticoagulated, into the first device 400a (CPD-1).

STEP 2: Process blood into components infranatant 910, and cloudy plasma 915 by centrifuging at 1900 g for 2 minutes. Extract plasma 915 using a syringe.

STEP 3: Load plasma 915 into the second device 400b (CPD-2). Process by centrifuging at 1900 g for 5 minutes.

STEP 4: Extract platelet poor plasma (PPP) 925 above platelet rich plasma (PRP) 920 from second device using a syringe.

STEP 5: Extract PRP 920 using a syringe.

### Example 5—Processing Blood, Double Spin

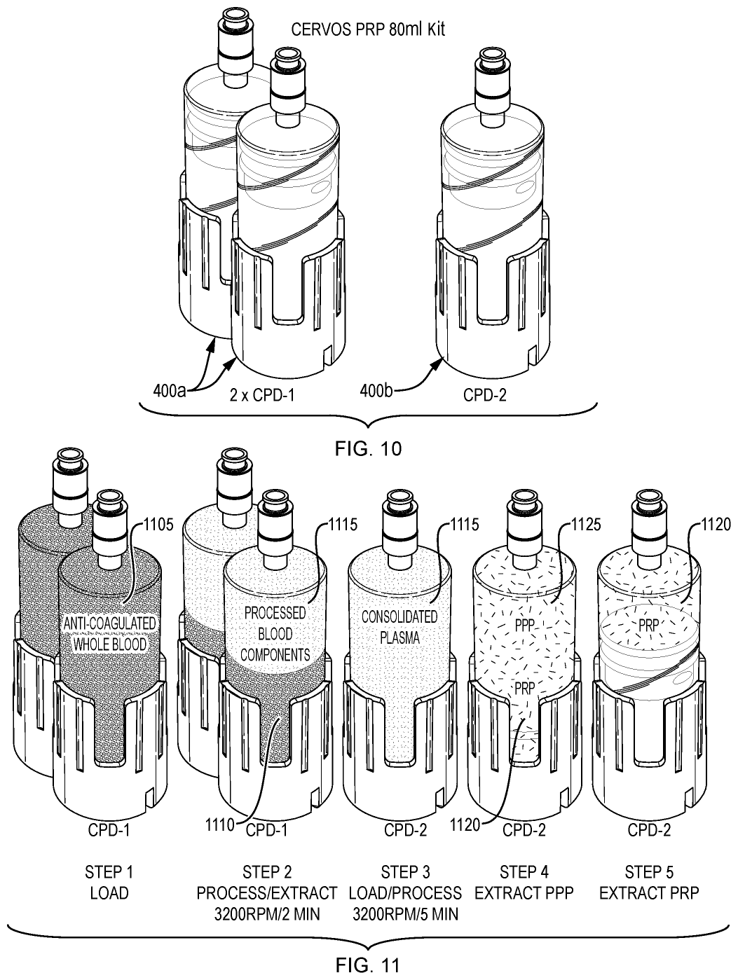

FIG. 10 illustrates another kit for processing blood that includes three devices, two devices 400a (2×CPD-1) plus one device 400b (CPD-2), for centrifuging a biological fluid allowing for larger volumes of the fluid, e.g., about 80 ml, to be processed. The devices are configured for use with a standard centrifuge.

FIG. 11 illustrates an example process of using the kit and devices of FIG. 10 to obtain platelet rich plasma (PRP) from blood using two centrifugation steps (double spin).

STEP 1: Load blood 1105, optionally anticoagulated, into each of the first devices 400a (CPD-1).

STEP 2: Process blood into components infranatant 1110 and cloudy plasma 1115 by centrifuging at 3200 RPM for 2 minutes (in standard centrifuge). Extract plasma 1115 from each device using one or more syringes.

STEP 3: Load plasma 1115 from each of the first devices into the second device 400b (CPD-2). Process by centrifuging at 3200 RPM for 5 minutes (in standard centrifuge).

STEP 4: Extract platelet poor plasma (PPP) 1125 above platelet rich plasma (PRP) 1120 from the second device using a syringe.

STEP 5: Extract PRP 1120 from the second device using a syringe.

Prior approaches to centrifuging physiological fluids have used a funnel-shaped insert positioned in a centrifugation container to separate cell fractions. Embodiments of the present invention do not require such an insert. Examples of prior approaches using inserts are described in the following published applications, the teachings of which are incorporated herein by reference in their entirety:

Cell separation methods and apparatus are described in International Application No. PCT/US2006/042237, filed on Oct. 27, 2006 and published on May 3, 2007 as WO2007/050986 A1. Cell concentration devices and methods are described in International Application No. PCT/US2014/013636, filed on Jan. 29, 2014 and published on Aug. 7, 2015 as WO2014/120797 A1. Apparatus and methods for aspirating and separating components of different densities from a physiological fluid containing cells are described in International Application No. PCT/US2010/036696, filed on May 28, 2010 and published on Dec. 2, 2010 as WO2010/138895 A2.

Physiological fluids, such as bone marrow, can be aspirated using double-cannula needle assemblies. Examples of such approaches are described in the following patent applications, the teachings of which are incorporated herein by reference in their entirety:

Apparatus and methods for aspirating and separating components of different densities from a physiological fluid containing cells are described in International Application No. PCT/US2010/036696, filed on May 28, 2010 and published on Dec. 2, 2010 as WO2010/138895 A2. Apparatus and methods for aspirating tissue are described in International Application No. PCT/US2013/067358, filed on Oct. 29, 2013 and published on May 8, 2014 as WO2014/070804 A1. An aspiration device and associated method including an introducer needle assembly, an aspiration needle assembly and a screw assembly are described in International Application No.: PCT/US2015/011614, filed on Jan. 15, 2015 and published on Jul. 23, 2015 as WO2015/109100 A1. An aspiration device and method including an introducer cannula, an aspiration cannula and a mechanism (e.g., a screw assembly) to move the cannulae are described in U.S. application Ser. No. 14/885,821, filed on Oct. 16, 2015 and published on Apr. 21, 2016 as US 2016/0106462 A1.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A device for centrifuging a physiological fluid, the device comprising:
a) a container including a tip, a base, and a barrel extending between the tip and the base to hold a physiological fluid;
b) a plunger positioned within the barrel of the container, the plunger including a concave collection surface facing the tip of the container and a gasket seal in sealing engagement with an inside wall of the barrel; and
c) a bottom support sized and shaped to support the base of the container at least partially within the bottom support for use in a centrifuge, the container being coupled to the bottom support using an interference fit.

2. The device of claim 1, further comprising a screen positioned in the barrel of the container between the plunger and the tip of the container.

3. The device of claim 1, wherein the bottom support includes a support base having a hole to provide access to the plunger of the container, the hole being threaded to accept a screw mechanism configured to apply a mechanical force to the plunger.

4. The device of claim 1, wherein the collection surface of the plunger and at least the barrel of the container are made of the same material.

5. The device of claim 4, wherein the material is polypropylene.

6. The device of claim 1, further comprising a top support sized and shaped to support the tip of the container at least partially within the top support for use in a centrifuge, the container being coupled to the top support using an interference fit.

7. The device of claim 1, wherein the container tip includes a luer port and wherein the device further comprises a cap to close the luer port.

8. The device of claim 1, wherein the barrel includes at least one protrusion and the bottom support includes at least one recess configured to receive the protrusion, the recess and the protrusion cooperating to provide a snap fit engagement between the container and bottom support when coupled.

9. The device of claim 8, wherein the at least one protrusion of the barrel is cantilevered, and wherein the recess is windowed to allow an external force to be applied to the protrusion to displace the protrusion from the recess, to cause disengagement of the snap fit.

10. The device of claim 1, wherein the plunger includes a rounded top rim encircling the concave surface, a rounded bottom rim, and a sidewall extending between the top rim and bottom rim, the sidewall including an annular recess to hold the gasket seal.

11. A method for centrifuging a physiological fluid, the method comprising:
a) holding a physiological fluid in a container, the container including a tip, a base and a barrel extending between the tip and the base, the container including a plunger positioned within the barrel, the plunger including concave collection surface facing the tip of the container and a gasket seal in sealing engagement with an inside wall of the barrel;
b) supporting the base of the container at least partially within a bottom support, the container being coupled to the bottom support using an interference fit; and
c) centrifuging the physiological fluid in the container supported by the bottom support.

12. The method of claim 11, wherein the collection surface of the plunger and at least the barrel of the container are made of the same material.

13. The method of claim 11, further comprising supporting the tip of the container with a top support at least partially within the top support, the container being coupled to the top support using an interference fit, and wherein the physiological fluid is centrifuged in the container supported by the top and bottom and supports.

14. The method of claim 11, wherein the physiological fluid is centrifuged in a first centrifugation with the base of the container away from a center of a centrifuge rotor.

15. The method of claim 14, further comprising extracting a first fraction of the physiological fluid from the container after the first centrifugation.

16. The method of claim 15, wherein a portion of the physiological fluid is further centrifuged in a second centrifugation with the tip of the container away from a center of a centrifuge rotor.

17. The method of claim 16, further comprising extracting second and third fractions of the physiological fluid from the container after the second centrifugation.

18. The method of claim 17, wherein the physiological fluid is adipose aspirate, the first fraction is a lipid layer, the second fraction is infranatant, and the third fraction is adipose graft tissue.

19. The method of claim 16, wherein the container tip includes a luer port and wherein the method further comprises closing the luer port with a cap prior to the second centrifugation.

20. The method of claim 15, further comprising:
a) transferring the first fraction of the physiological fluid to a second container, the second container including a tip, a base and a barrel extending between the tip and the base, the second container including a plunger positioned within the barrel, the plunger including a concave collection surface facing the tip of the second container and a gasket seal in sealing engagement with an inside wall of the barrel; and
b) in a second centrifugation, centrifuging the first fraction of the physiological fluid in the second container supported by a second bottom support.

21. The method of claim 20, further comprising extracting second and third fractions of the physiological fluid from the second container after the second centrifugation.

22. The method of claim 21, wherein the physiological fluid is blood, the first fraction is plasma, the second fraction is platelet poor plasma (PPP), and the third fraction is platelet rich plasma (PRP).

23. The method of claim 11, wherein the physiological fluid is blood, and wherein the blood is centrifuged without adding anticoagulant to the blood prior to centrifugation.

24. The method of claim 11, further comprising positioning a screen in the barrel of the container between the plunger and the tip of the container.

25. The method of claim 11, wherein the bottom support includes a support base having a hole to provide access to the plunger of the container, and further comprising applying a mechanical force to the plunger with a screw mechanism threaded through the hole, the applied mechanical force assisting in extracting a fraction of the physiological fluid from the container.

26. A method for centrifuging a physiological fluid using the device of claim 1, the method comprising:
a) loading the physiological fluid into the container of the device; and
b) centrifuging the physiological fluid in the container supported by the bottom support of the device.

27. The method of claim 26, wherein the physiological fluid is blood, and wherein the blood is centrifuged without adding anticoagulant to the blood prior to centrifugation.
